(19) United States
(12) Patent Application Publication
(10) Pub. No.: US 2005/0257347 A1
(43) Pub. Date: Nov. 24, 2005

(54) Striate-body fixation fixture, wire-harness protector and fixation clip

(75) Inventor: Hirokazu Tsuchiya, Shizuoka-ken (JP)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: YAZAKI CORPORATION

(21) Appl. No.: 11/125,167

(22) Filed: May 10, 2005

(30) Foreign Application Priority Data
May 11, 2004 (JP) P2004-140820

(51) Int. Cl. A61F 2/30

(52) U.S. Cl. 24/1

(57) ABSTRACT

A striate-body fixation fixture includes a base plate a first side and a second side opposite to each other. The fixture includes a positioning plate included in the base plate for mounting the striate body in position on the first side of the base plate. The fixture includes first and second insertion holes extending through the base plate and opposed to each other with respect to the positioning plate. The fixture includes a guide wall defining at least one of the first and second insertion holes and approaching the other insertion hole for guiding a band to secure the striate body on the fixture as the guide wall extends toward the second side of the base plate.

### CROSS-REFERENCE TO RELATED APPLICATIONS

This application is based upon and claims the benefit of priority from Japanese Patent Application No. 2004-140820 filed on May 11, 2004; the entire contents of which are incorporated herein by reference.

### BACKGROUND OF THE INVENTION

The invention relates to a striate-body fixation fixture for mainly securing a striate-body, such as wire harness, by use of a band clamp, a wire-harness protector having the fixation plate and a wire-harness fixation clip.

In general, when it is required to fix a wire harness to any member of a vehicle, a band clamp (otherwise known as “binding band”) is employed as such fixation means (e.g. Japanese Patent Application Laid-open No. 2000-253536).

For example, in order to fix a wire harness to a fixation plate for a wire-harness protector by use of a band clamp, there are proposed a variety of methods. Note, the fixation plate is provided with two insertion holes. In one method, it is first performed to mount the wire harness on the fixation plate. Next, one end portion of the band clamp is inserted through one insertion hole from the upside of the fixation plate. Subsequently, from the underside of the fixation plate, the end portion of the band clamp is inserted through the other insertion hole upwardly. Then, the end portion passing through the other insertion hole upwardly is wound around the outer circumference of the wire harness to successively engage in a locking hole formed at the base of the band clamp. In this way, the fixture of the wire harness to the fixation plate is completed.

The fixation plate, however, is an element having insertion holes formed for passage of a band clamp merely, and it is difficult for an operator to insert the band clamp into the insertion hole of the fixation plate from the underside.

### SUMMARY OF THE INVENTION

In the above-mentioned situation, it is an object of the invention to provide a striate-body fixation fixture facilitating insertion of the end portion of the band clamp, a wire-harness protector and a wire-harness fixing clamp both using such a fixation plate.

In order to achieve the above object, according to the first aspect of the invention provides a striate-body fixation fixture. The fixture includes a base plate having a first side and a second side opposite to each other. The fixture includes a positioning plate included in the base plate for mounting the striate body in position on the first side of the base plate. The fixture includes first and second insertion holes extending through the base plate and opposed to each other with respect to the positioning plate. The fixture includes a guide wall defining at least one of the first and second insertion holes and approaching the other insertion hole for guiding a band to secure the striate body on the fixture as the guide wall extends toward the second side of the base plate.

The guide wall extends obliquely relative to a thickness direction of the base plate.

The band is inserted through a first insertion hole from the first side to the second side of the base plate, and is inserted through a second insertion hole from the second side to the first side of the base plate. The band is wounded around the striate body, thereby securing the striate-body to the fixture.

The guide wall includes first and second guide walls defining the first and second insertion holes, respectively

The guide wall is curved toward the other insertion hole.

The positioning plate includes a structure to prevent the striate body from transverse displacement.

The positioning plate includes a curved wall recessed from the first side of the base plate. The curved wall defines a recess for locating the striate body therein to prevent the striate body from transverse displacement.

The curved wall has a bottom wall serving as the guide wall.

At least one of the first and second insertion holes extends obliquely to the thickness direction of the base plate.

The striate-body includes a wire-harness

A wire-harness protector includes a wire-harness protective wall formed by the striate-body fixation fixture.

A wire-harness fixation clip provided, on the striate-body fixation fixture, with a clip for engagement with a panel hole.

These and other objects and features of the invention will become more fully apparent from the following description and appended claims taken in conjunction with the accompany drawings.

### BRIEF DESCRIPTION OF THE DRAWINGS

FIG. 1 is a sectional view of a substantial part heat of a fixation plate in accordance with an embodiment of the invention;

FIG. 2 is a perspective view of the substantial part heat of the fixation plate of FIG. 1; and

FIGS. 3A to 3E are schematic perspective views of fixation plates in various modifications for the embodiment of FIG. 1.

### DETAILED DESCRIPTION OF THE PREFERRED EMBODIMENT

Referring to accompanying drawings, an embodiment of the invention will be described below.

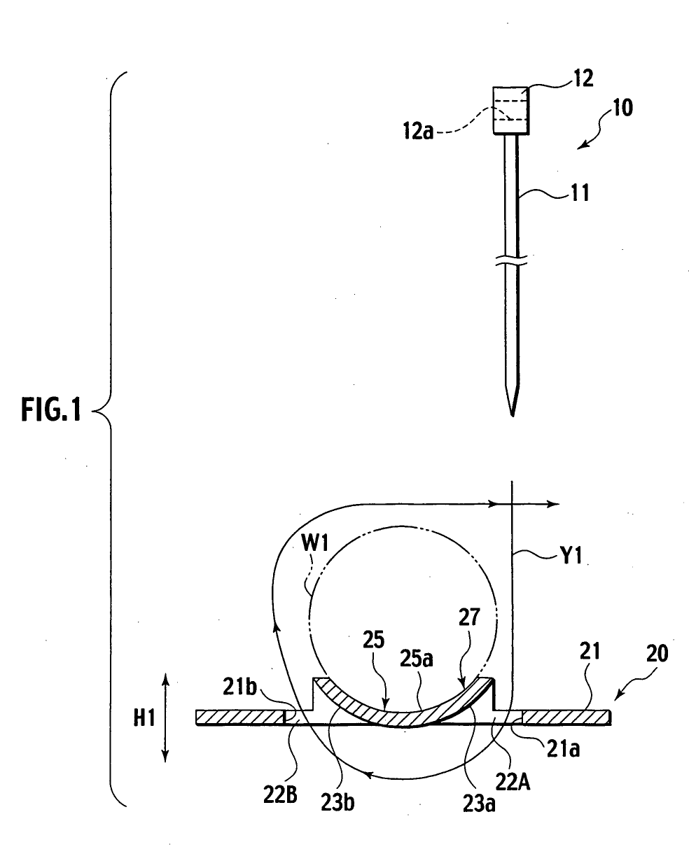

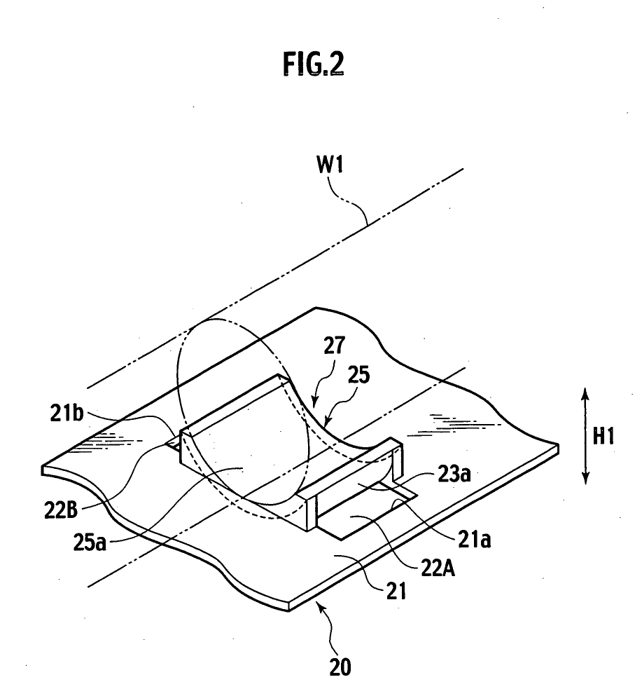

In FIGS. 1 and 2, a fixation plate 20 of the embodiment is applied to one protective wall of a wire-harness protector.

This fixation plate 20 has first and second sides opposite to each other in a thickness direction H1 thereof. The fixation plate 20 includes a level part 21 as a base plate. The fixation plate 20 includes a positioning part 27 as a positioning plate arranged inside the level part 21 to mount a wire harness (striate body) W1 thereon on the first side. The positioning part 27 has, on both sides thereof, two rectangular first and second insertion holes 22A, 22B through the level part 21 in a thickness direction H1 of the fixation plate 20. The first and second insertion holes 22A, 22B are separated from each other in a transverse direction of the wire harness W1. Additionally, the first and second insertion holes 22A, 22B have, outside thereof, outer sidewalls 21a, 21b extending in the thickness direction H1, respectively. In order to fix the wire harness W1 to the fixation plate 20, the end portion of a band 11 of a band clamp 10 is first inserted through the first insertion hole 22A from the upside or the first side. After this insertion, the end portion of the band clamp 10 is inserted through the second insertion hole 22B from the underside upwardly. Then, the end portion of the band 11 passing through the second insertion hole 22B is wound around the outer circumference of the wire harness W1 to successively engage in a locking hole 12a of a base 12 of the band clamp 10. This way secures the wire harness W1 on the fixation plate 20.

The positioning part 27 for mounting the wire harness W1 includes a curved wall 25 that is recessed downwardly from above view or the first side. The upper recess of the curved wall 25 operates as means 25a for preventing the wire harness W1 from transverse displacement. The curved wall 25 includes, at the bottom on both sides thereof, first and second guide walls 23a, 23b serving as guides for band clamp 10. These guide walls 23a, 23b constitute the inner sidewalls of the first and second insertion holes 22A, 22B, respectively. The first and second guide walls 23a, 23b are curved toward the second and first insertion holes, respectively as they 23a, 23b extend from the upside or the first side toward the underside or the second. That is, the first and second guide walls 23a, 23b are tangentially oblique to the thickness direction H1 of the level part 21. In other words, the first or second guide wall 23a or 23b is curved to approach the first or second insertion hole 22B or 22A as extending from the first side (wire-harness side) of the fixation plate 20 equipped with the wire harness W1 toward the second side of the fixation plate 20.

The feature of the fixation plate 20 provides the first and second guide walls 23a, 23b having curved surfaces, which constitute respective inner sidewalls of two first and second insertion holes 22A, 22B. Note that, if forming a loop by the band 11, then a side face positioned inside the loop corresponds to the above inner sidewalls of the first and second insertion holes 22A, 22B. During insertion of the end portion of the band 11 through the first insertion hole 22A (or the second insertion hole 22B), the first guide wall 23a (or the second guide wall 23b) operates to guide the insertion of the end portion of the band 11 into the first insertion hole 22A (or the second insertion hole 22B). Further, one of the first and second guide walls 23a, 23b operates to guide the inserted band 11 to the other of the first and second guide walls 23a, 23b.

A method of fixing the wire harness W1 to the fixation plate 20 will be described below. First of all, with the wire harness W1 mounted on the curved wall 25, the end portion of the band 11 of the band clamp 10 is moved along a route shown with arrow Y1. In detail, the end portion of the band 11 is first inserted through first insertion hole 22A from the first side or upside and successively inserted through the second insertion hole 22B upwardly from the second side or underside. Next, the end portion of the band 11 inserted upwardly through the second insertion hole 22B is wound around the outer circumference of the wire harness W1 and further engage in the locking hole 12a of the base 12 of the band clamp 10. This engagement allows the wire harness W1 to be fixed on the fixation plate 20.

As mentioned above, the second insertion hole 22B is formed with the second guide wall 23b with a curved surface as the inner sidewall. During the above-mentioned securing operation of the band clamp 10, the first guide wall 23a has a guide operation that allows the end portion of the band 11 to easily pass through the insertion hole 22B from the underside or second side to the upside or first side, thus enhancing operator's workability. Additionally, the wire harness W1 is mounted on the upper concave part 25a of the curved wall 25, thus preventing the wire harness W1 from transverse displacement, with the wire harness W1 stably fixed. The provision of the first guide walls 23a, 23b in the inner sidewalls of the first and second insertion holes 22A, 22B allows an operator to easily insert the end portion of the band 11 into either one of the first and second holes 22A and 22B from the underside or second side to the upside or first side. In other words, an operator may insert the band 11 into either of the first and second insertion holes 22A and 22B at first.

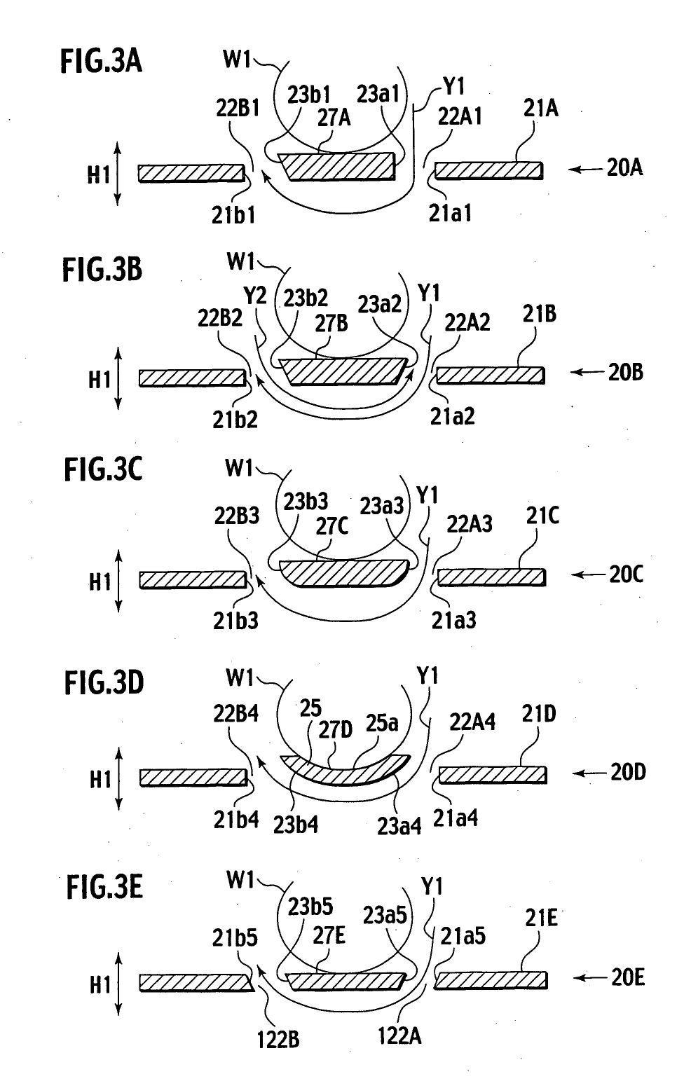

FIGS. 3A to 3D schematically show a variety of fixation plates in modifications of the invention. Note that, throughout the modifications, elements similar to those of FIG. 1 are indicated with similar reference numerals, respectively.

In the modification of FIG. 3A, a fixation plate 20A includes two first and second insertion holes 22A1, 22B1. Further, the second insertion hole 22B1 is provided, on an inner sidewall thereof, with a second guide wall 23b1. The second guide wall 23b1 is formed so as to extend straightly and obliquely to the thickness direction H1 of a level part 21A. In detail, the second guide wall 23b1 of the second insertion hole 22B1 is formed so as to approach the first insertion hole 22A1 as extending from the first side (i.e. the wire-harness's side) to the second side of the level part 21A.

In the modification of FIG. 3B, a fixation plate 20B includes two first and second insertion holes 22A2, 22B2. Both of the first and second insertion holes 22A2, 22B2 are provided, on their respective inner sidewalls, with first and second guide walls 23a2, 23b2, respectively. The first and second guide walls 23a2, 23b2 are formed so as to extend straightly and obliquely to the thickness direction H1 of a level part 21B. In detail, the first guide wall 23a2 of the first insertion holes 22A2 (or the second guide wall 23b2 of the second insertion hole 22B2) is formed so as to approach the second insertion hole 22B2 (or first insertion hole 22A2) as extending from the first side (i.e. the wire-harness's side) to the second side of the level part 21B.

In the modification of FIG. 3C, a fixation plate 20C includes two first and second insertion holes 22A3, 22B3. Both of the first and second insertion holes 22A3, 22B3 are provided, on their respective inner sidewalls, with first and second guide walls 23a3, 23b3 in the form of curved surfaces, respectively. The first guide wall 23a3 of the first insertion holes 22A3 (or the second guide wall 23b3 of the second insertion hole 22B3) is curved so as to approach the second insertion hole 22B3 (or first insertion hole 22A3) as extending from the first side (i.e. the wire-harness's side) to the second side of a level part 21C. The first and second guide walls 23a3, 23b3 are tangentially oblique to the thickness direction H1 of the level part 21C.

In the modification of FIG. 3D, a fixation plate 20D is similar to the fixation plate 20 of the above-mentioned embodiment shown in FIGS. 1 and 2, in the provision of a curved wall 25 for mounting a wire harness W1 thereon. The fixation plate 20D includes two first and second insertion holes 22A4, 22B4. The curved wall 25 of this modification is provided, on both sides of the bottom surface thereof, with first and second guide walls 23a4, 23b4. The upper concave part 25a of the curved wall 25 operates as means for preventing the wire harness W1 (i.e. a striate body) from transverse displacement.

In the modification of FIG. 3E, a fixation plate 20E includes a positioning part 27E flush with a level part 21E. Additionally, the fixation plate 20E is provided, as the insertion holes, with first and second guide slots 122A, 122B oblique to the thickness direction H1 of the fixation plate 20E. The inner sidewalls of the first and second guide slots 122A, 122B constitute first and second guide walls 23a5, 23b5 extending obliquely to the thickness direction H1 of the level part 21E, respectively. The guide walls 21a5, 21b5 as the outer sidewalls of the first and second guide slots 122A, 122B extend obliquely to the thickness direction H1 of the level part 21E, functioning as guides for the band clamp. The first and second guide walls 23a5, 21b5 (or the guide walls 23b5, 21b5) are formed so as to approach the second slot 122B (or the first slot 122A) as extending from the first side (i.e. the wire-harness's side) to the second side of the level part 21E.

According to this modification, it is possible to make the positioning part 27E of the fixation plate 20E in level with the level part 21E.

In the above-mentioned embodiment, the upper concave part 25a of the curved wall 25 constitutes means for preventing the wire harness (i.e. the striate body) from transverse displacement. Alternatively, the fixation plate may be provided with the other means for preventing the wire harness from transverse displacement, for example, a plurality of ribs projecting from the fixation plate.

Additionally, in a further modification, there may be attached, at appropriate positions of the fixation plate 20, clips for engagement with attaching holes formed in the plate 20, providing wire-harness fixation clamps.

In the above description, the adjective terms “upper” and “lower” are nothing but terms expressing one side of the fixation plate for mounting the wire harness W1 (i.e. an operator's view side) and the other side of the fixation plate, respectively.

Finally, it will be understood by those skilled in the art that the foregoing descriptions are nothing but one embodiment and various modification of the disclosed fixation plate and therefore, various changes and modifications may be made within the scope of claims.

According to the invention, the band is guided by the guide wall to be inserted through at least one of the first and second insertion holes. The guide facilitates insertion of the band, and enhancing workability.

Location of guide wall in proximity to the positioning plate allows the band to be guided from one of the first and second insertion holes to the other hole.

The curved guide wall achieves smooth guide operation.

The structure prevents the striate-body from transverse displacement, thus stably fixing the striate-boy.

The curved wall of the positioning plate prevents the striate-body from transverse displacement and functions as a guide during insertion of the band, thus achieving stable securing and smooth winding operation of the striate body.

The oblique insertion hole allows for a level configuration of the base plate and the positioning plate.

1. A striate-body fixation fixture comprising:
a base plate having a first side and a second side opposite to each other;
a positioning plate included in the base plate for mounting the striate body in position on the first side of the base plate;
first and second insertion holes extending through the base plate and opposed to each other with respect to the positioning plate; and
a guide wall defining at least one of the first and second insertion holes and approaching the other insertion hole for guiding a band to secure the striate body on the fixture as the guide wall extends toward the second side of the base plate.

2. The striate-body fixation fixture of claim 1,
wherein the guide wall extends obliquely relative to a thickness direction of the base plate.

3. The striate-body fixation fixture according to claim 1,
wherein the band is inserted through a first insertion hole from the first side to the second side of the base plate, and is inserted through a second insertion hole from the second side to the first side of the base plate,
wherein the band is wounded around the striate-body, thereby securing the striate body to the fixture.

4. The striate-body fixation fixture of claim 1,
wherein the guide wall includes first and second guide walls defining the first and second insertion holes, respectively.

5. The striate-body fixation fixture according to claim 1,
wherein the guide wall is curved toward the other insertion hole.

6. The striate-body fixation fixture according to claim 1,
wherein the positioning plate includes a structure to prevent the striate body from transverse displacement.

7. The striate-body fixation fixture according to claim 6,
wherein the positioning plate includes a curved wall recessed from the first side of the base plate,
wherein the curved wall defines a recess for locating the striate body therein to prevent the striate body from transverse displacement.

8. The striate-body fixation fixture according to claim 7,
wherein the curved wall has a bottom wall serving as the guide wall.

9. The striate-body fixation fixture according to claim 1,
wherein at least one of the first and second insertion holes extends obliquely to the thickness direction of the base plate.

10. The striate-body fixation fixture according to claim 1,
wherein the striate-body includes a wire-harness.

11. A wire-harness protector including a wire-harness protective wall formed by the striate-body fixation fixture of claim 1.

12. A wire-harness fixation clip provided, on the striate-body fixation fixture of claim 1, with a clip for engagement with a panel hole.
